(19) United States
(12) Patent Application Publication
(10) Pub. No.: US 2020/0115754 A1
(43) Pub. Date: Apr. 16, 2020

(54) AGE DETERMINATION OF HUMAN INDIVIDUAL

(71) Applicant: BEIERSDORF AG, Hamburg (DE)

(72) Inventors: Marc WINNEFELD, Wedel (DE); Elisabeth WURZER, Hamburg (DE); Boris KRISTOF, Halstenbek (DE); Lars KADERALI, Greifswald (DE)

(21) Appl. No.: 16/621,295

(22) PCT Filed: Jun. 12, 2018
(86) PCT No.: PCT/EP2018/065460

§ 371 Date: Dec. 11, 2019

(30) Foreign Application Priority Data
Jun. 12, 2017 (EP) 17175573.9
Aug. 24, 2017 (DE) 10 2017 119 427.4

(51) Int. Cl. C12Q 1/6881; C12Q 1/6883

(52) U.S. Cl. CPC C12Q 1/6881; C12Q 2600/148; C12Q 2600/154; C12Q 1/6883

(57) ABSTRACT

The present invention relates to a method for determining the biological age of human skin comprising providing human skin cells, determining a methylation level of at least two CpG-dinucleotides of a specific region of at least one chromosome of said skin cells and determining the biological age of said skin cells by comparing said determined methylation level with empirically determined data representing a correlation between the methylation level of the CpG-nucleotide and the chronological age of at least one human individual.

### FIELD OF THE INVENTION

The present invention relates to the determination of biological age of human skin by analysing the epigenetic pattern of particular sites of the DNA. The methylation levels at selected CpG sites of a skin sample are evaluated to determine the biological age of human skin accurately. The measured biological age is then correlated to the chronological age with a mean absolute error of less than 5.05 years.

### BACKGROUND OF THE INVENTION

Epigenetic changes of DNA are known to occur over time and have been recognised as indicators of the extent of the aging process in human individuals. In particular, the extent of methylation of CpG dinucleotides has been identified as being related to age.

Methods of biological age determination by the analysis of various CpG-dinucleotides are known. In Weidner et al, Genome Biology, 2014 15R24, for example, 102 age-related CpG sites in blood have been identifies as suitable for estimating the biological age of an individual. The described method uses data from 3 CpG sites to achieve a mean absolute deviation from the chronological age of less than 5 years. EP 2 711 431 also analyses blood cells and predicts the chronological age of the subject based on 6 of the 102 identified age-related CpG sites. The prediction based on 6 CpG sites to determine the biological age was found to differ from the chronological age by about 4.53 years to as much as 10.30 years. In another study, a difference of ±3.6 years between the chronological age and biological age was found by Horvath, Genome Biology 2013, 14:R115 by characterising 353 CpG sites from blood cells.

The use of blood samples in the analysis of epigenetic change is suboptimal due to the various cell types found in the blood that have differing DNA methylation patterns. Furthermore, bacterial and viral infections can significantly alter the cell composition in blood, which can shift the methylation pattern.

Human skin has also been used as a model for the analysis of age-related epigenetic changes and correlated to the chronological age of an individual. The known predictor according to Horvath, Genome Biology 2013, 14:R115 was used on human epidermis in Bormann et al, Aging Cell, 2016, 15, page 563 to 571 and the average absolute prediction error was found to be 14.5 years. This shows that the published predictor underestimates the chronological age of epidermis samples. In Bormann et al, Aging Cell, 2016, 15, page 563 to 571, a further predictor was developed using the methylation of 450,000 CpG dinucleotides, which were simultaneously analysed by an array-based approach. The biological age determined using the method therein was within an error of less than 5.25 years of the chronological age. However the drawback of this method is the large number of sites that need to be analysed, which is time consuming and cost intensive.

It is therefore desirable to provide improved and/or alternative methods to determine the biological age of an individual. Preferably, it is desired to provide a method that uses a reduced number of CpG sites.

### SUMMARY OF THE INVENTION

The present invention is defined in the appended claims.

In accordance with a first aspect, there is provided a method for determining the biological age of human skin comprising:
a) determining a methylation level of at least two CpG-dinucleotides of human skin cells; and
b) determining the biological age of said skin cells by comparing said determined methylation level with empirically determined data representing a correlation between the methylation level of said CpG-nucleotides and the chronological age of at least one human individual.

In accordance with a second aspect, there is provided a method of testing an active agent comprising the method according to the first aspect, further comprising the steps of:
c) contacting the skin cells with an active agent;
d) determining the biological age of the skin cells of step c) according to the method of the first aspect; and
e) comparing the biological age determined in steps a) and b) with the biological age determined in step d).

In accordance with a third aspect, there is provided the use of a nucleic acid molecule for determining the biological age of human skin according to the method of the first aspect, wherein said nucleic acid molecule comprises at least one nucleotide sequence selected from the group consisting of:
a) a nucleotide sequence comprising at least one sequence selected from the group consisting of SEQ ID NO: 1 to SEQ ID NO: 205;
b) a nucleotide sequence which differs from the nucleotide sequence of a) by replacement of at most 10% of the nucleotides, preferably at most 5 of the nucleotides, except for said CpG-dinucleotide;
c) a nucleotide sequence which corresponds to the complementary strand of the nucleotide sequence a) and/or b).

In accordance with a fourth aspect, there is provided a computer-readable medium having stored computer-executable instructions for causing a computer to perform a method for determining the biological age of human skin comprising:
a) inputting at least two values of a determined methylation level of at least two

CpG-dinucleotides of human skin cells;
b) comparing said value of the determined methylation level with stored data representing a correlation between the methylation level of said CpG-dinucleotide and the chronological age of at least one human individual; and
c) displaying the biological age.

In accordance with a fifth aspect, there is provided a kit for determining the biological age of human skin according to the method of the first aspect, comprising at least one oligonucleotide primer for amplifying and/or sequencing at least two CpG-nucleotides of at least one nucleotide sequence selected from the group consisting of SEQ ID NO: 1 to SEQ ID NO: 205.

Certain embodiments of the present invention may provide one or more of the following advantages:
desired ease of sample collection from the individual;
desired speed of obtaining bioinformatic data;
desired ease of obtaining bioinformatic data;
desired cost-effective data collection;
desired accuracy of age prediction.

The details, examples and preferences provided in relation to any particular one or more of the stated aspects of the present invention apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE DRAWINGS

The invention will further be illustrated by reference to the following figures:

FIG. 1 table of CpG sites and nucleotide sequences for SEQ ID No: 1 to 6;

FIG. 2 table of CpG sites and nucleotide sequences for SEQ ID No: 7 to 12;

FIG. 3 table of CpG sites and nucleotide sequences for SEQ ID No: 13 to 18;

FIG. 4 table of CpG sites and nucleotide sequences for SEQ ID No: 19 to 24;

FIG. 5 table of CpG sites and nucleotide sequences for SEQ ID No: 25 to 30;

FIG. 6 table of CpG sites and nucleotide sequences for SEQ ID No: 31 to 36;

FIG. 7 table of CpG sites and nucleotide sequences for SEQ ID No: 37 to 42;

FIG. 8 table of CpG sites and nucleotide sequences for SEQ ID No: 43 to 48;

FIG. 9 table of CpG sites and nucleotide sequences for SEQ ID No: 49 to 54;

FIG. 10 table of CpG sites and nucleotide sequences for SEQ ID No: 55 to 60;

FIG. 11 table of CpG sites and nucleotide sequences for SEQ ID No: 61 to 66;

FIG. 12 table of CpG sites and nucleotide sequences for SEQ ID No: 67 to 72;

FIG—table of CpG sites and nucleotide sequences for SEQ ID No: 73 to 78;

FIG. 14 table of CpG sites and nucleotide sequences for SEQ ID No: 79 to 84;

FIG. 15 table of CpG sites and nucleotide sequences for SEQ ID No: 85 to 90;

FIG. 16 table of CpG sites and nucleotide sequences for SEQ ID No: 91 to 96;

FIG. 17 table of CpG sites and nucleotide sequences for SEQ ID No: 97 to 102;

FIG. 18 table of CpG sites and nucleotide sequences for SEQ ID No: 103 to 108;

FIG. 19 table of CpG sites and nucleotide sequences for SEQ ID No: 109 to 114;

FIG. 20 table of CpG sites and nucleotide sequences for SEQ ID No: 115 to 120;

FIG. 21 table of CpG sites and nucleotide sequences for SEQ ID No: 121 to 126;

FIG. 22 table of CpG sites and nucleotide sequences for SEQ ID No: 127 to 132;

FIG. 23 table of CpG sites and nucleotide sequences for SEQ ID No: 133 to 138;

FIG. 24 table of CpG sites and nucleotide sequences for SEQ ID No: 139 to 144;

FIG. 25 table of CpG sites and nucleotide sequences for SEQ ID No: 145 to 150;

FIG. 26 table of CpG sites and nucleotide sequences for SEQ ID No: 151 to 156;

FIG. 27 table of CpG sites and nucleotide sequences for SEQ ID No: 157 to 162;

FIG. 28 table of CpG sites and nucleotide sequences for SEQ ID No: 163 to 168;

FIG. 29 table of CpG sites and nucleotide sequences for SEQ ID No: 169 to 174;

FIG. 30 table of CpG sites and nucleotide sequences for SEQ ID No: 175 to 180;

FIG. 31 table of CpG sites and nucleotide sequences for SEQ ID No: 181 to 186;

FIG. 32 table of CpG sites and nucleotide sequences for SEQ ID No: 187 to 192;

FIG. 33 table of CpG sites and nucleotide sequences for SEQ ID No: 193 to 198;

FIG. 34 table of CpG sites and nucleotide sequences for SEQ ID No: 199 to 204;

FIG. 35 table of CpG sites and nucleotide sequences for SEQ ID No: 205;

FIG. 36 graph represents the change in methylation with age for two exemplary CpGs markers: cg06335867 and cg13848598;

FIG. 37 graph represents a correlation of chronological age with biological age using the markers cg06335867 and cg13848598, using the formula: 70.002+8.992*cg06335867+12.998*cg13848598

It is understood that the following description and references to the figures concern exemplary embodiments of the present invention and shall not be limiting the scope of the claims.

### DETAILED DESCRIPTION

The present invention is based on the finding that the methylation level of selected CpG nucleotides can determine the biological age of human skin.

An increase in age (aging) is the process of becoming older. Age may be viewed in various ways, such as chronological age and biological age.

As used herein, the term “chronological age” refers to the amount of time that has elapsed since the birth of an individual. The term “individual” as used herein refers to a human individual.

As used herein, the “biological age” refers to physical changes in an individual, such as the extent of epigenetic changes of DNA. The extent of epigenetic changes of DNA is determined in skin samples. The biological age is determined from skin samples and correlates to the biological age of the individual. The biological age as disclosed herein is determined using the method of the first aspect of this invention.

In particular, the methylation level of specific CpG-dinucleotides are assessed as part of this invention. The methylation level can be determined, for example by methylation specific PCR, sequence analysis of bisulfite treated DNA, CHIP-sequencing (Illumina Methylation BeadChip Technology), molecular inversion probe assay, Methyl-CAP-sequencing, Next-Generation-sequencing, COBRA-Assay, methylation specific restriction patterns or MassARRAY assay.

In certain embodiments the biological age of the human skin is equal to the biological age of the individual.

Biological age may be influenced by many parameters such as genetic background, disease and lifestyle.

The biological age may differ from the chronological age. The biological age may provide an indication of the health of the individual when compared with the chronological age. If, for example, the biological age is lower than the chronological age, it may be concluded that on a biological level signs of aging are less predominant than expected. This may indicate that the individual in good health. Alternatively, if the biological age is higher than the chronological age, it may be taken as sign that the individual is in poor health.

In certain embodiments, the human skin cells used according to the method of the invention are obtained by harvesting the entire skin sample required from the individual. In certain embodiments, the human skin cells used according to the method of the invention are obtained by culturing the skin cells using an in vitro method.

Harvesting a sample from the individual may be carried out using suction blistering, punch biopsy, shave biopsy or during any surgical procedure such as plastic surgery, lifting, grafting, or the like.

The skin samples may be taken from the epidermis or dermis.

Human skin cells may be cultured from a small sample of skin cells harvested from an individual. The harvested human skin cells may be grown in vitro in a vessel such as a petri dish in a medium or substrate that supplies essential nutrient.

The human skin cells used may be a mixture of harvested cells and cultured cells.

In certain embodiments, the specific region of the chromosome comprising CpG-dinucleotides according the invention may be coding regions or non-coding regions. In certain embodiments, the CpG dinucleotides may be present in a coding region and/or non-coding region. The CpG dinucleotides may be found in a single specific region or in different specific regions.

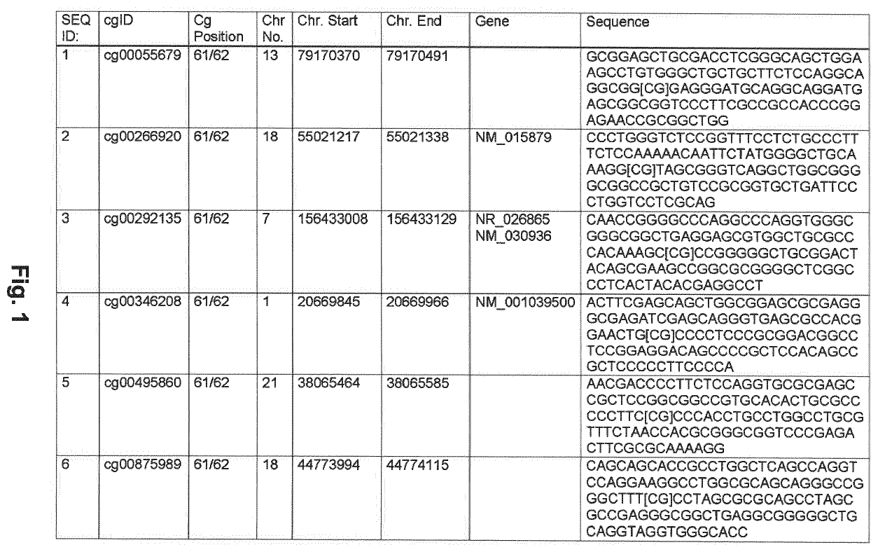

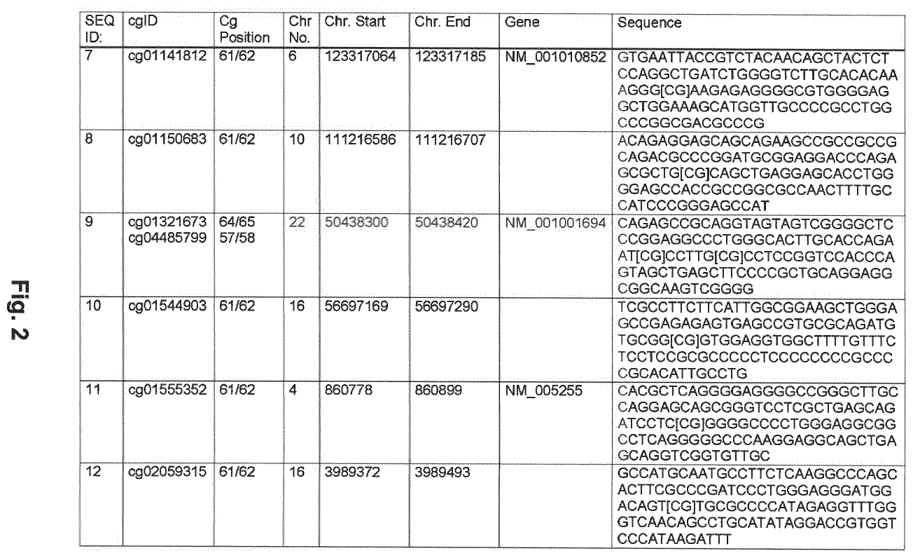

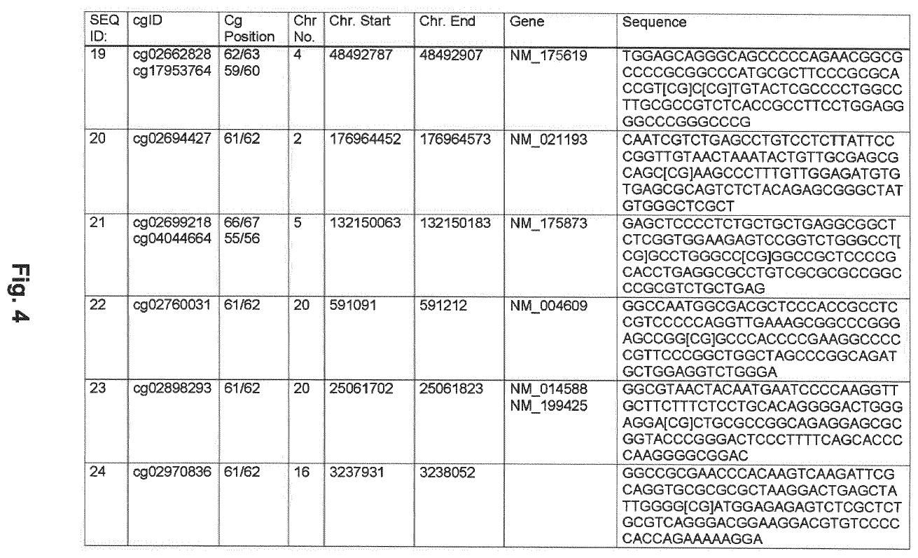

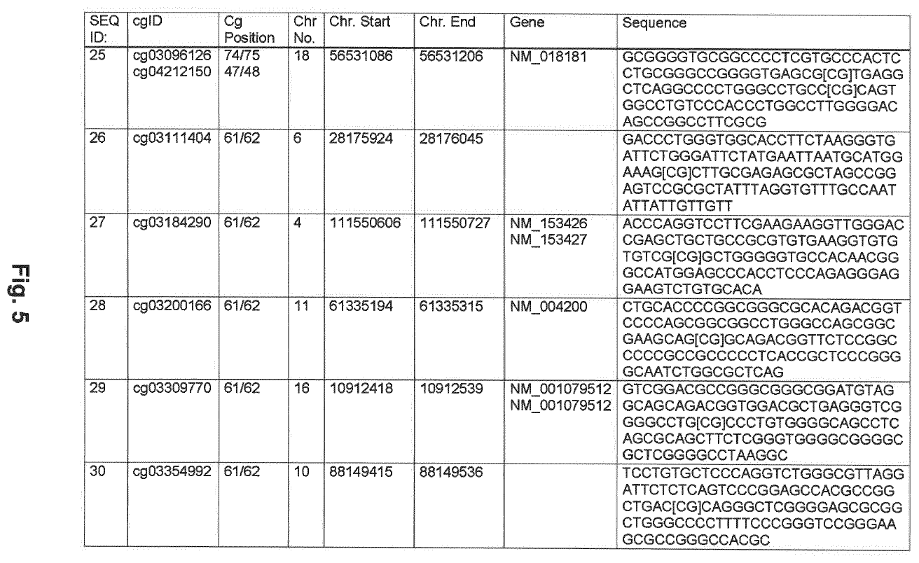

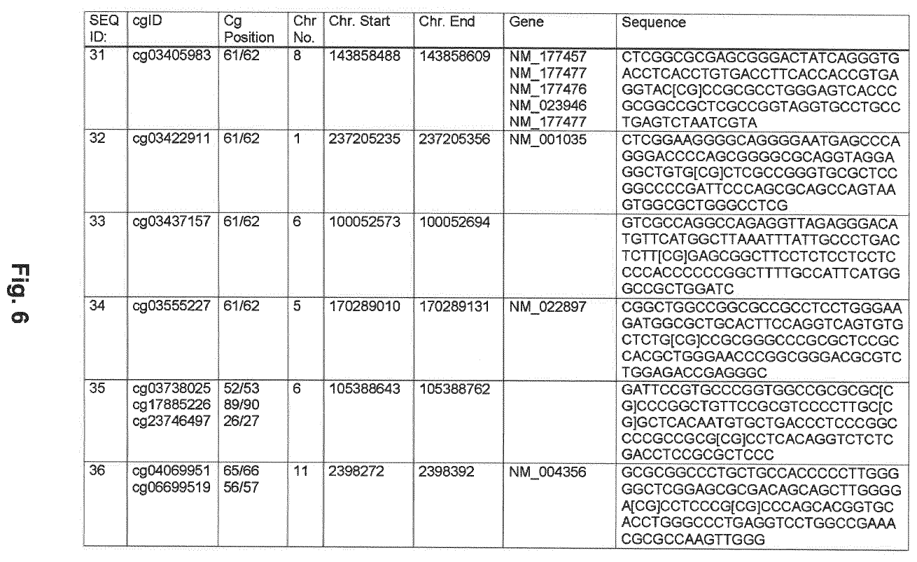

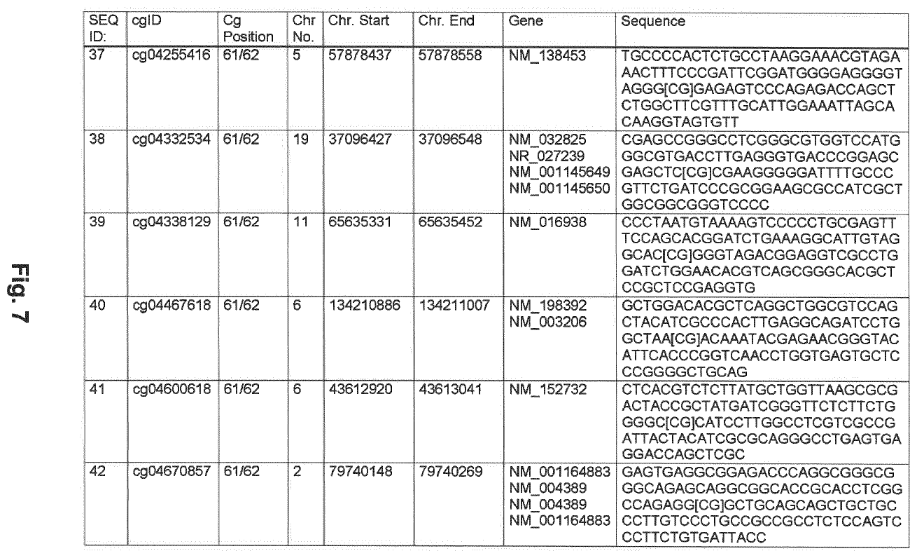

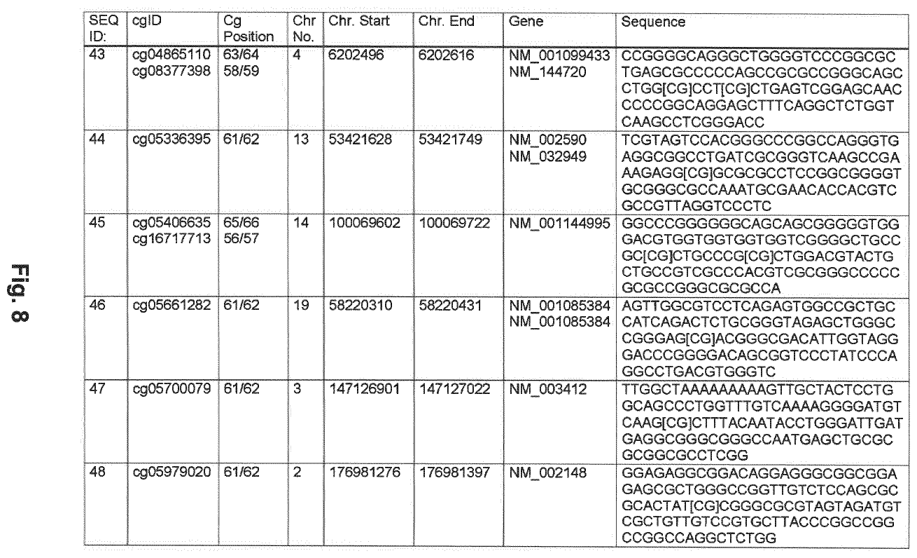

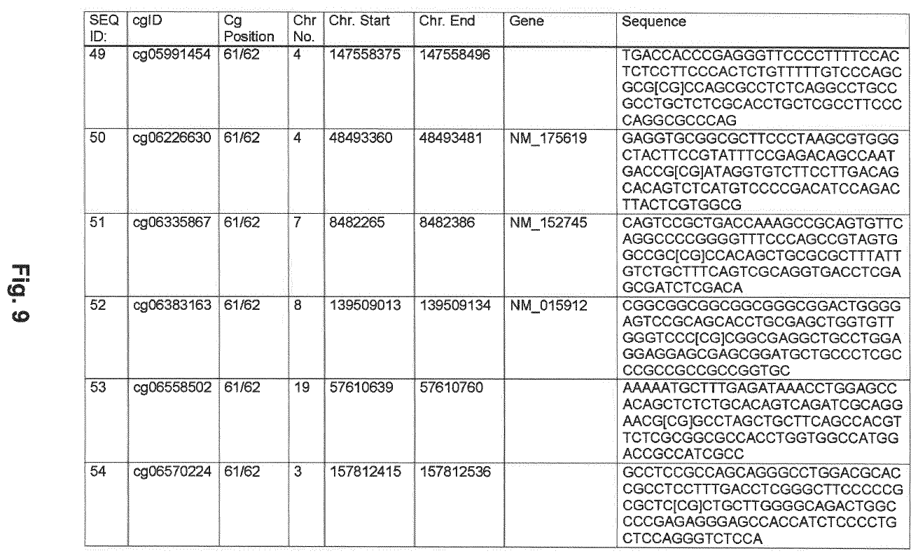

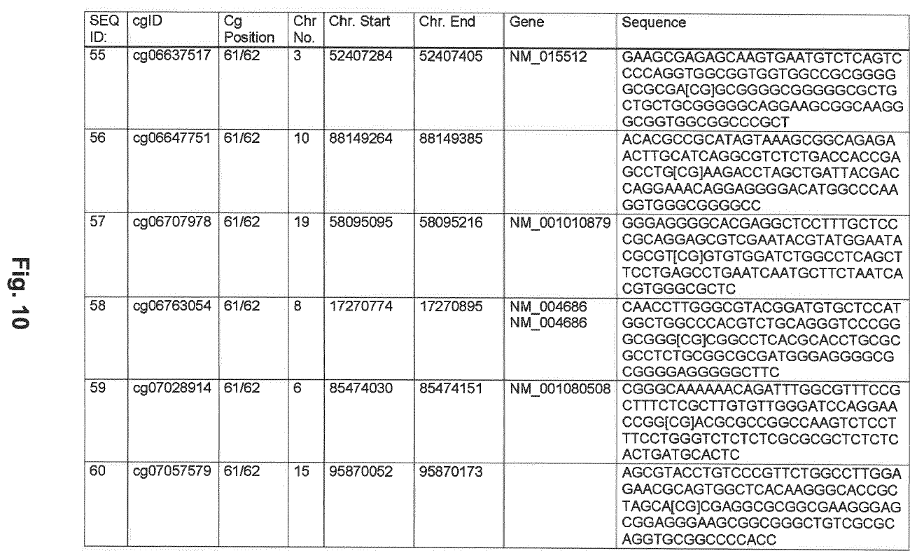

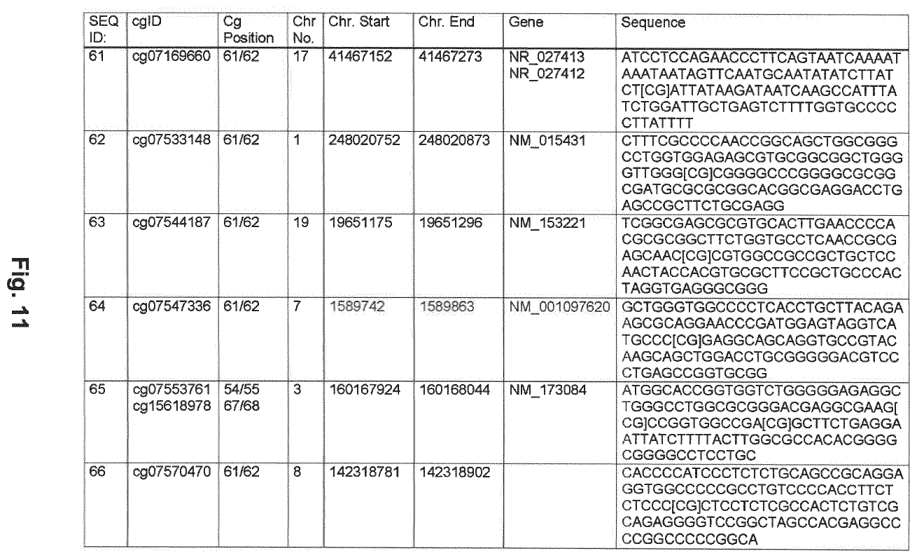

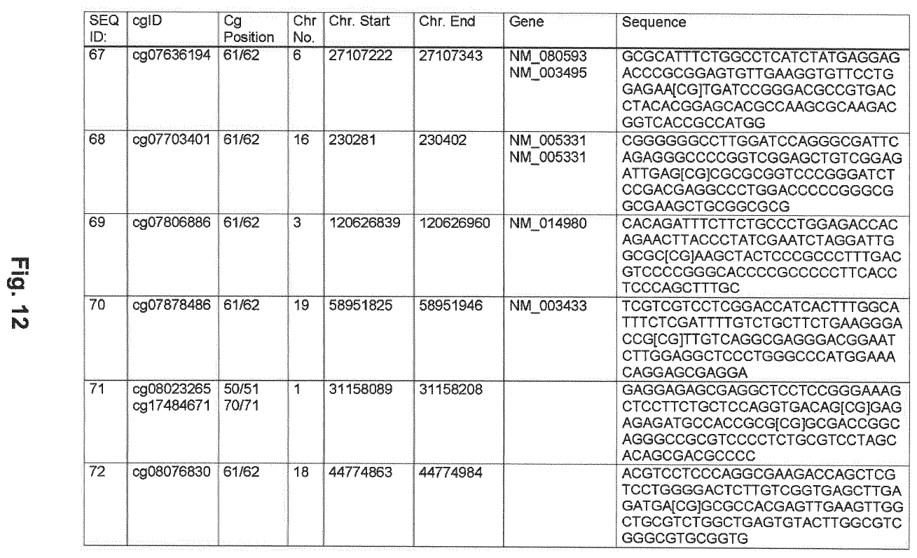

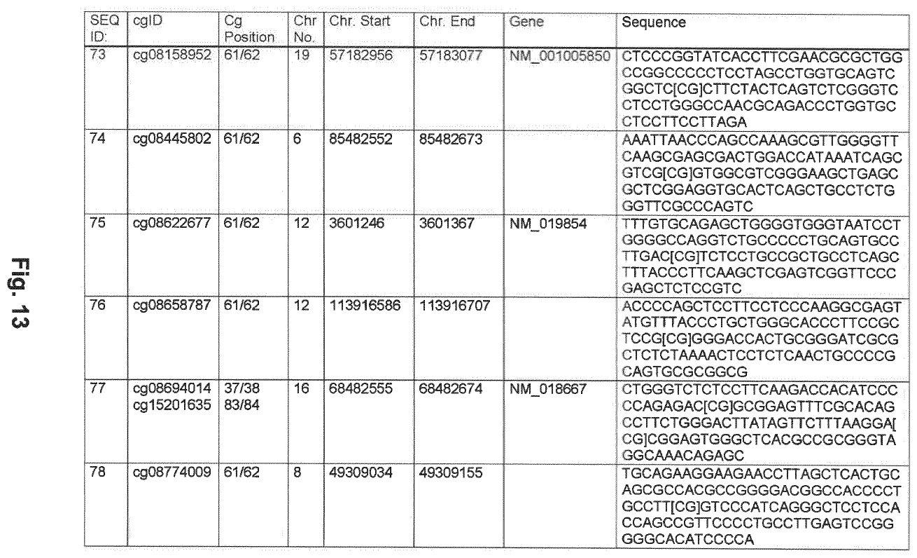

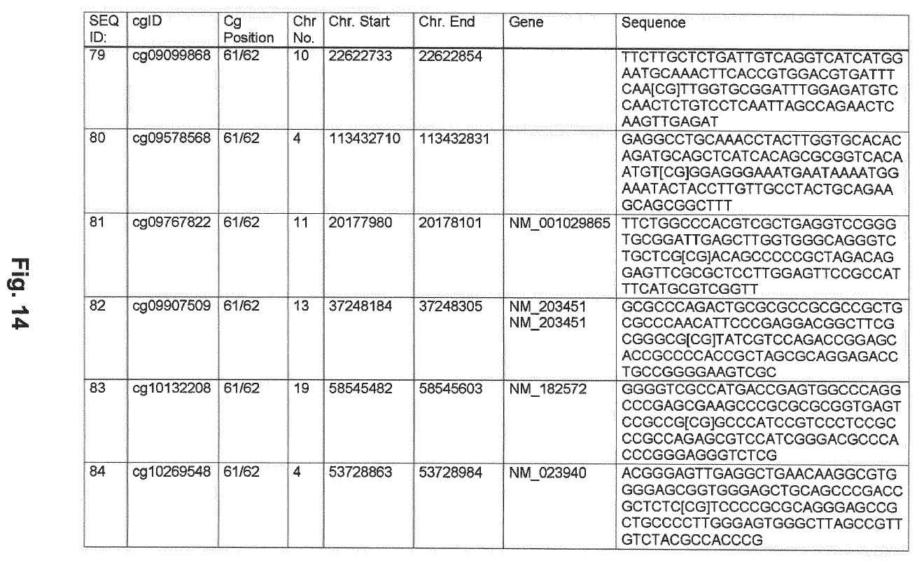

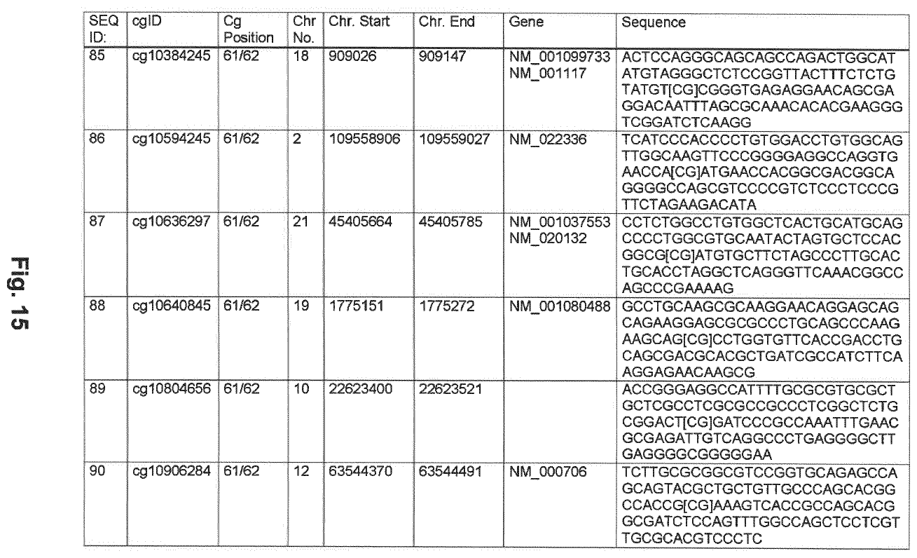

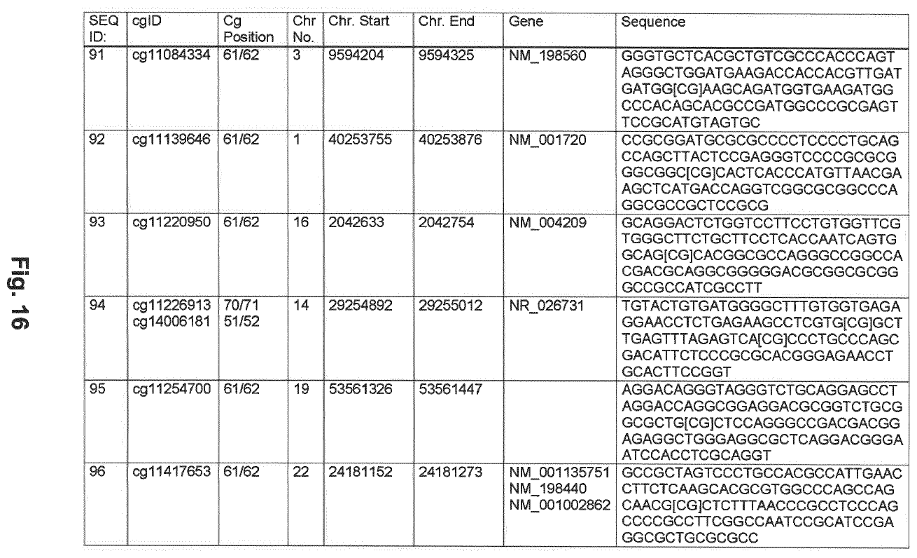

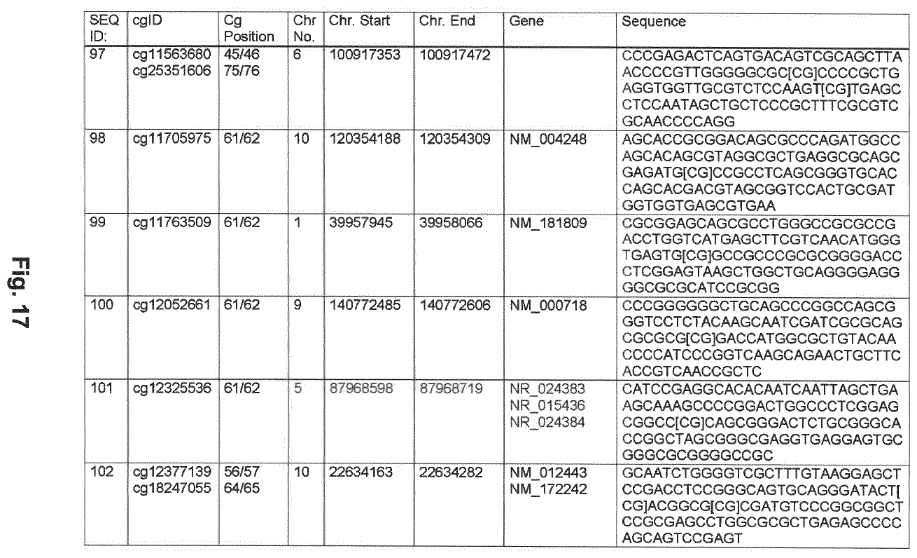

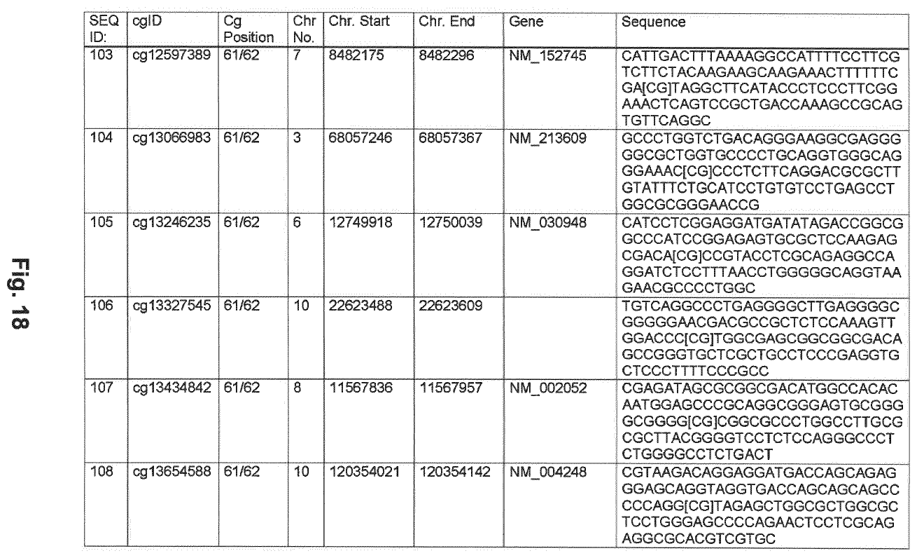

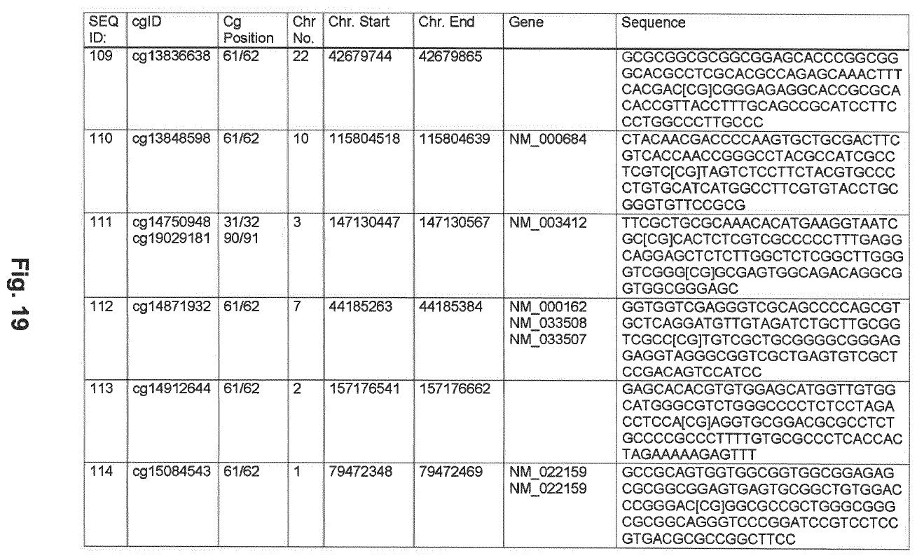

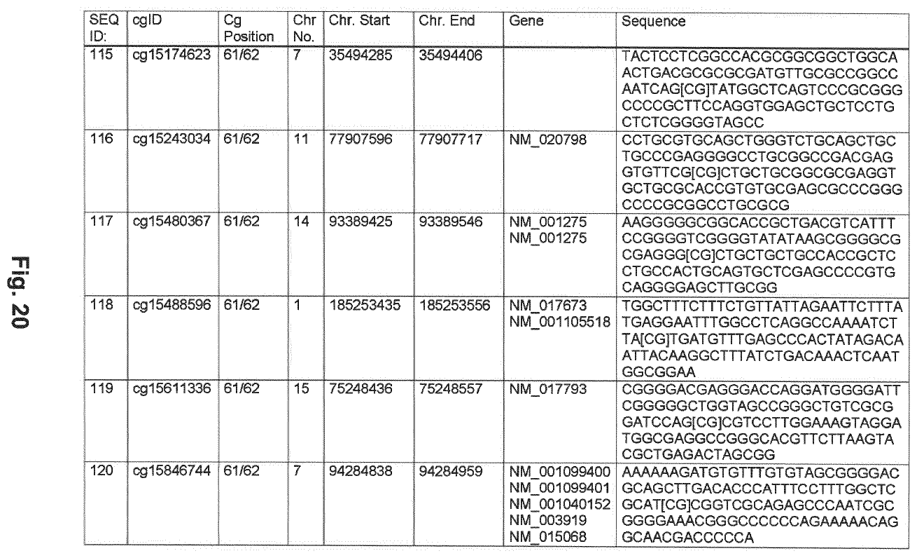

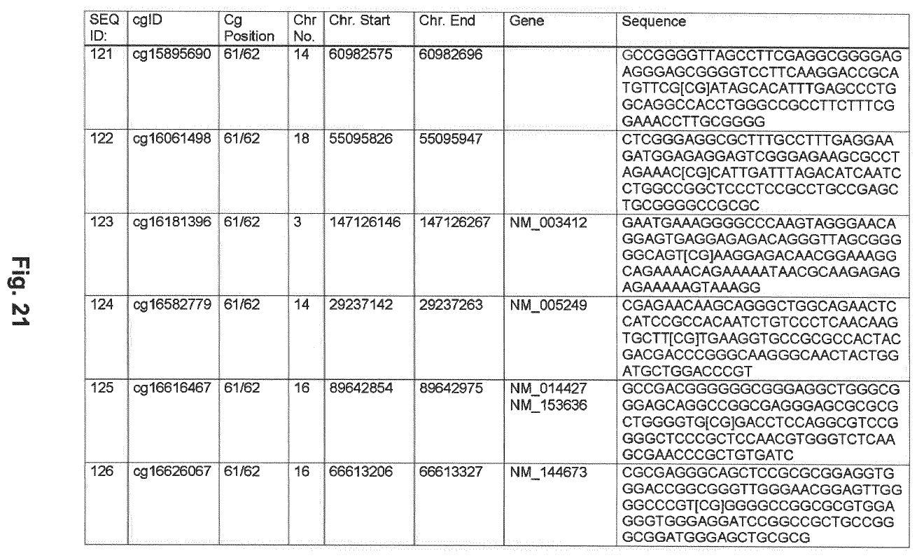

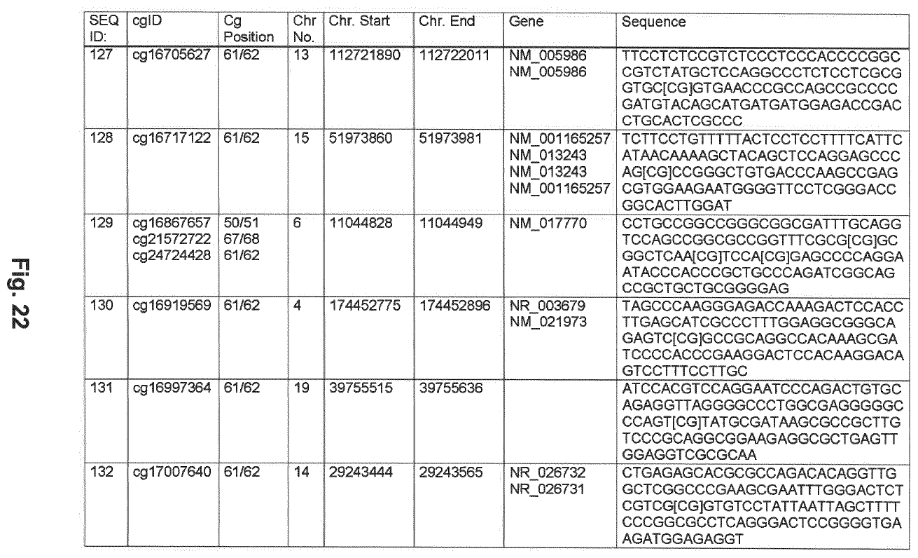

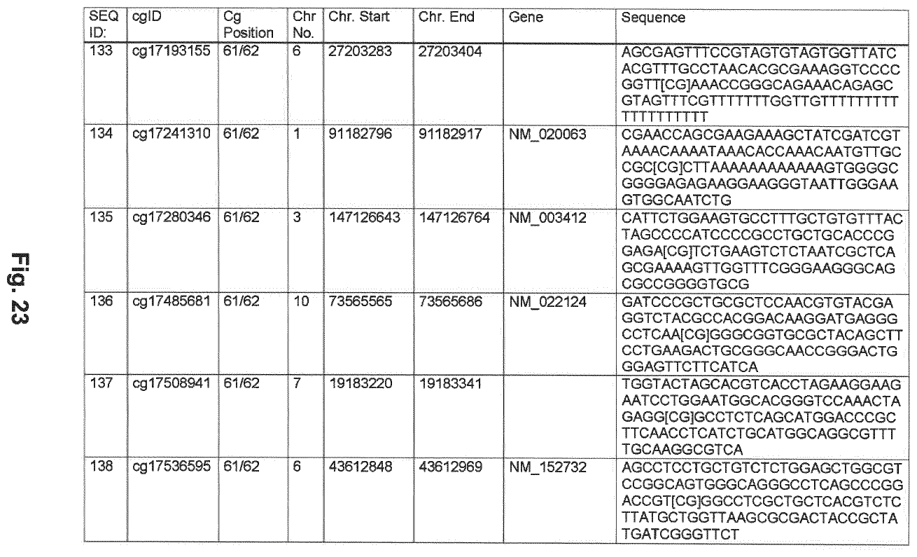

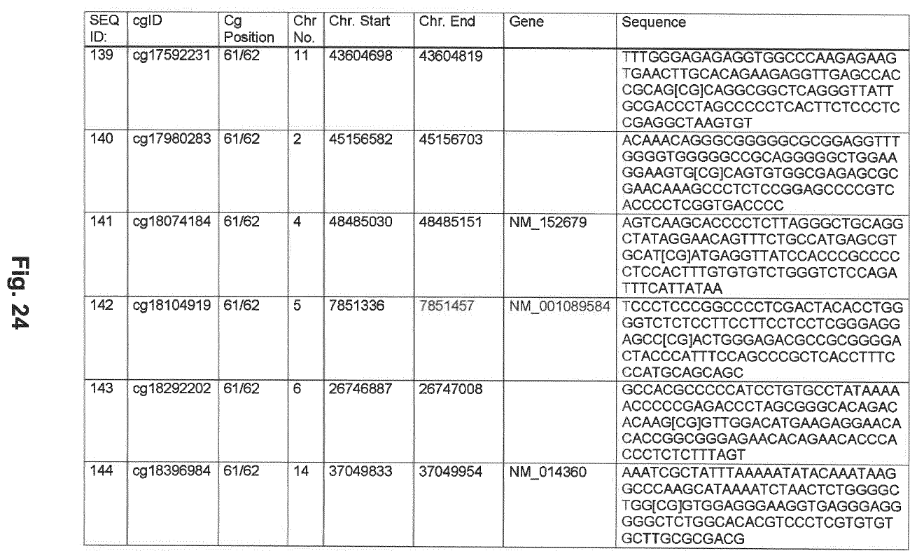

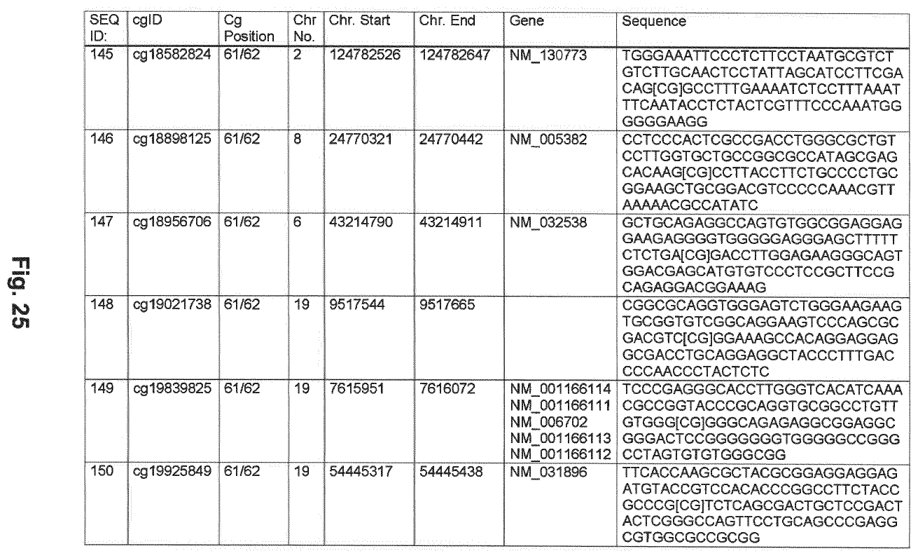

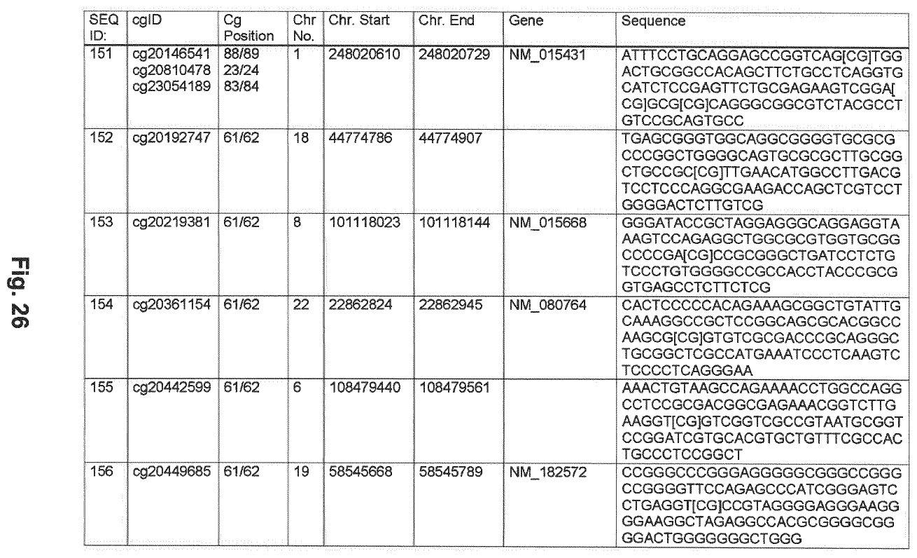

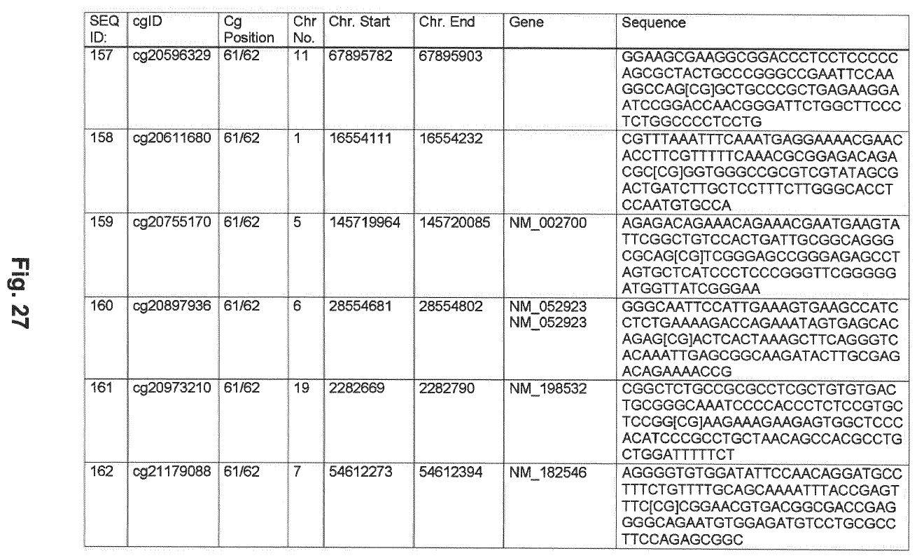

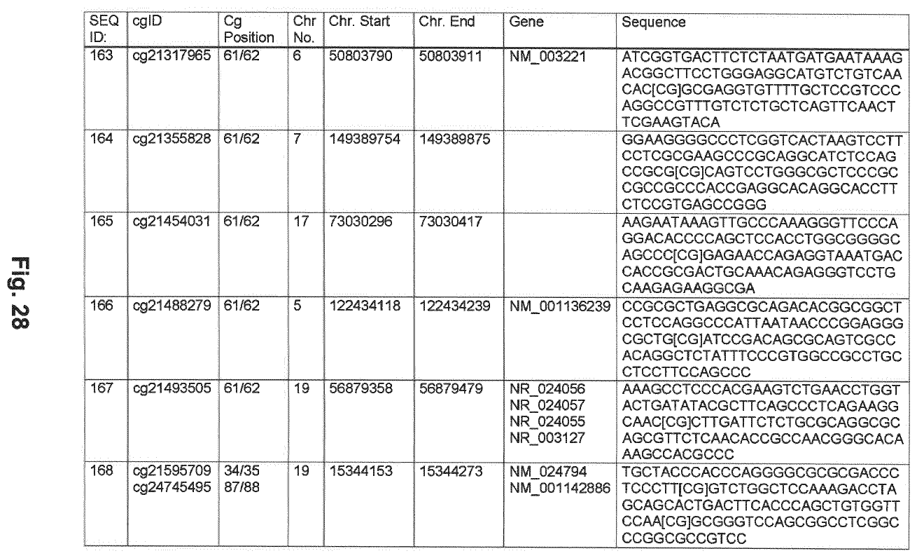

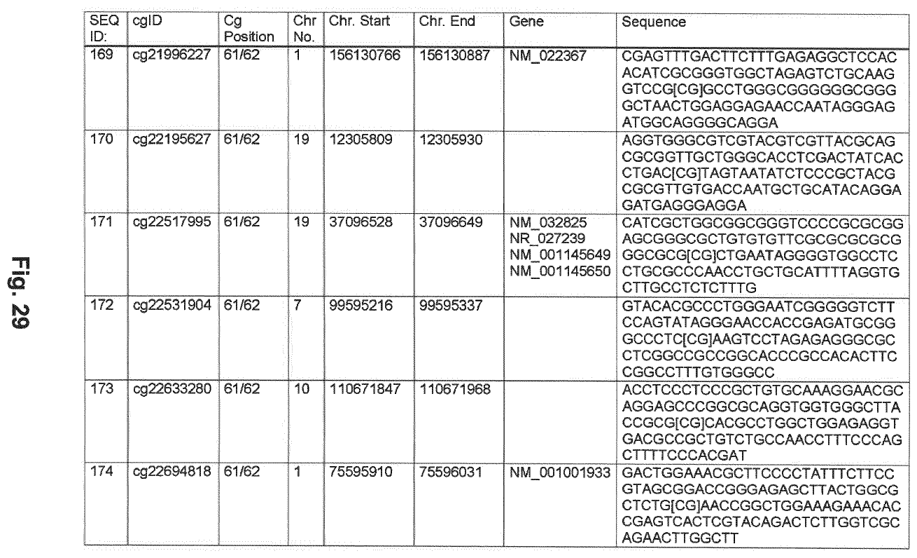

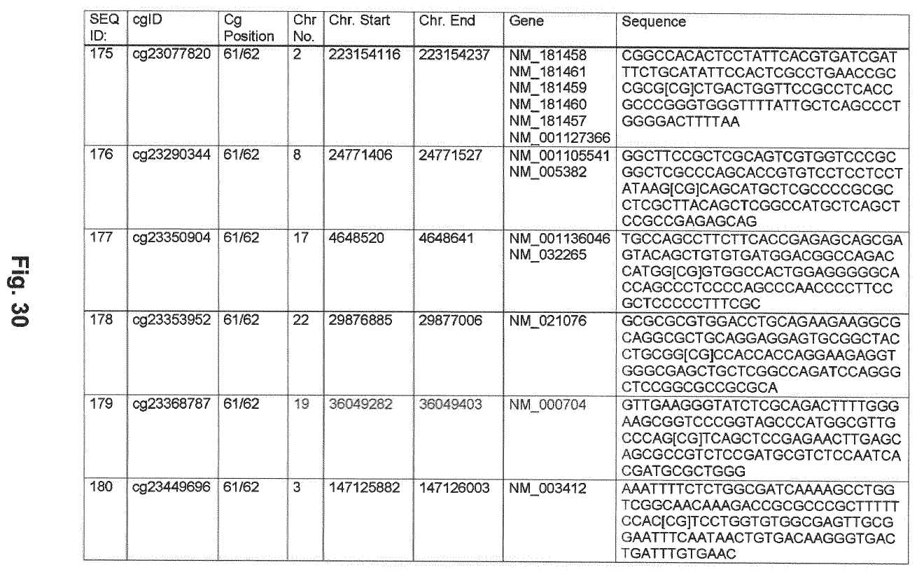

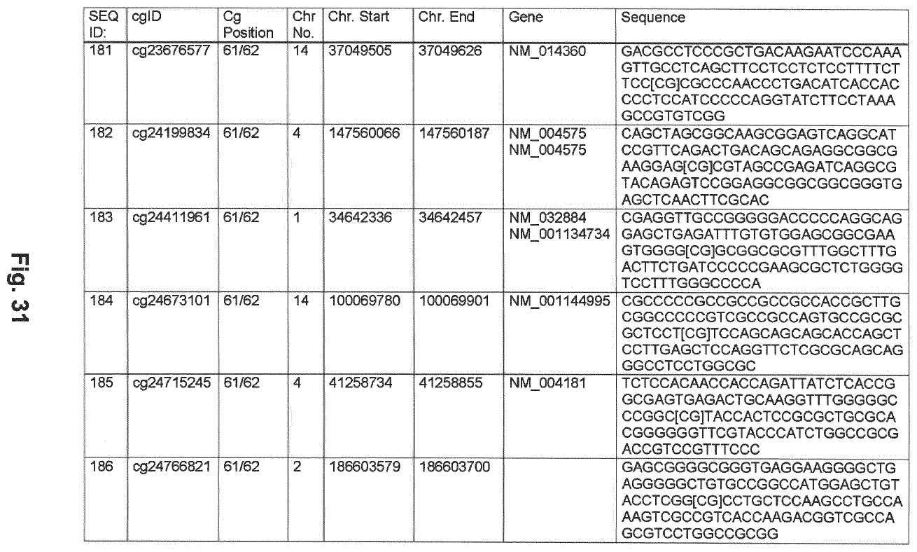

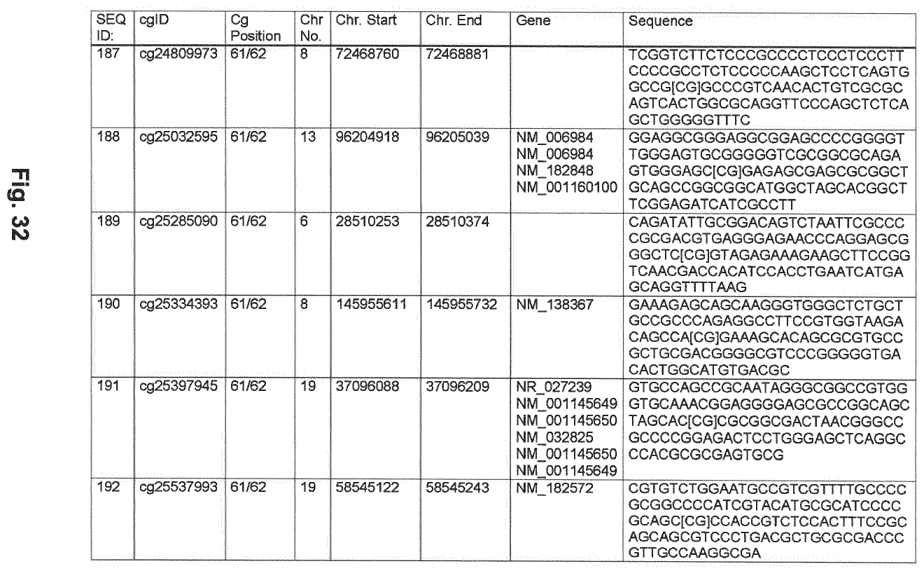

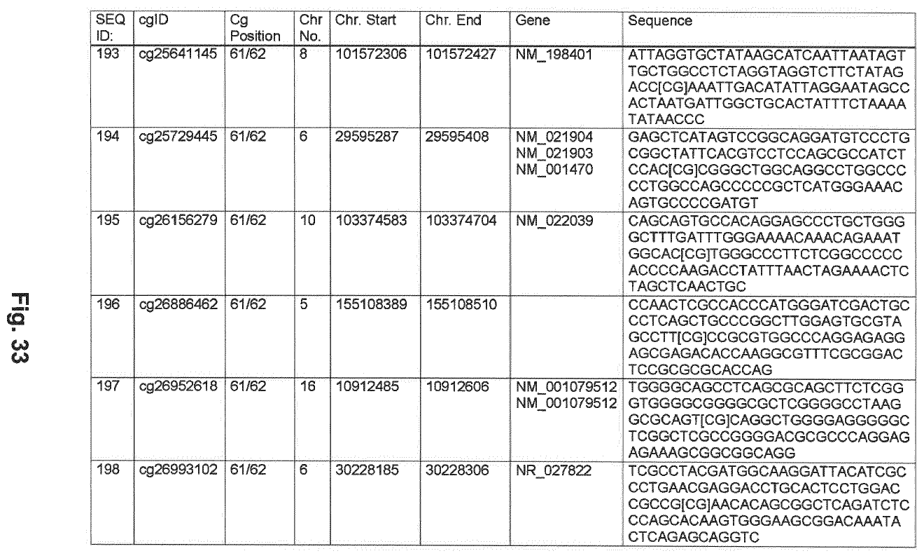

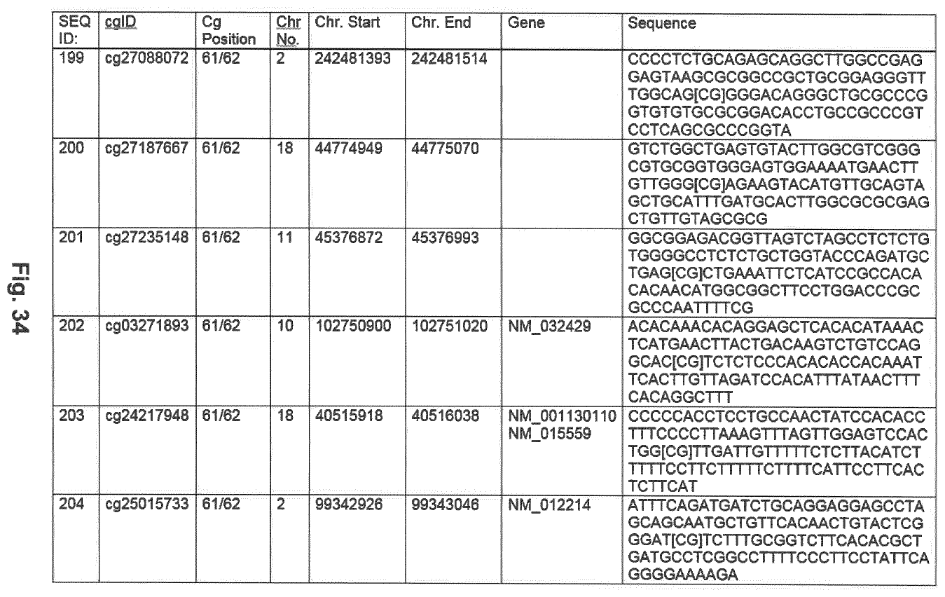

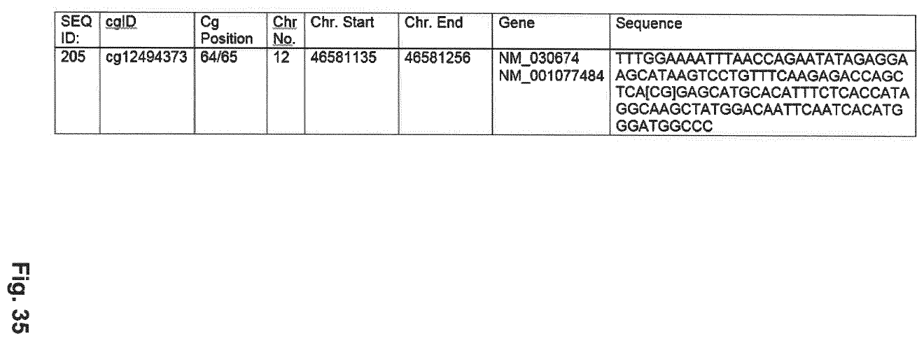

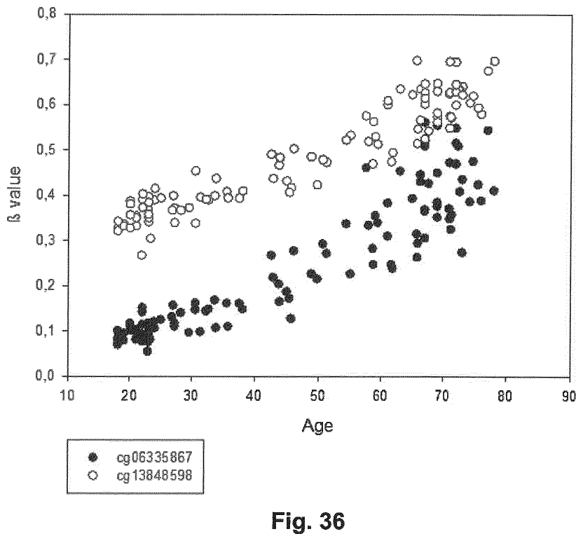

In certain embodiments, the specific region of the chromosome comprises at least one nucleotide sequence from SEQ ID NO. 1 to SEQ ID NO. 205 as shown in FIGS. 1 to 35. The correlation of each SEQ ID to the CpG dinucleotide according to the invention is shown in FIGS. 1 to 35.

In certain embodiments the method of the first aspect may involve determining the methylation level of two CpG dinucleotides. The two CpG dinucleotides whose methylation level is determined may be on the same nucleotide sequence, such as, for example, on SEQ ID NO. 9, SEQ ID NO. 13, SEQ ID NO. 16, SEQ ID NO. 19, SEQ ID NO. 21, SEQ ID NO. 25, SEQ ID NO. 35, SEQ ID NO. 36, SEQ ID NO. 43, SEQ ID NO. 45, SEQ ID NO. 65, SEQ ID NO. 71, SEQ ID NO. 77, SEQ ID NO. 94 97, SEQ ID NO. 102, SEQ ID NO. 111, SEQ ID NO. 129, SEQ ID NO. 151 and SEQ ID NO. 168. The two CpG dinucleotides may be on different dinucleotide sequences, each selected independently from SEQ ID NO. 1 to SEQ ID NO. 205.

In certain embodiments the method of the first aspect may involve determining the methylation level of three CpG dinucleotides. In certain embodiments the method of the first aspect may involve determining the methylation level of four CpG dinucleotides. In certain embodiments the method of the first aspect may involve determining the methylation level of five CpG dinucleotides. In certain embodiments the method of the first aspect may involve determining the methylation level of six CpG dinucleotides. In certain embodiments the method of the first aspect may involve determining the methylation level of seven CpG dinucleotides. In certain embodiments the method of the first aspect may involve determining the methylation level of eight CpG dinucleotides. In certain embodiments the method of the first aspect may involve determining the methylation level of nine CpG dinucleotides. In certain embodiments the method of the first aspect may involve determining the methylation level of ten CpG dinucleotides. In certain embodiments the method of the first aspect may involve determining the methylation level at least two and no more than three CpG dinucleotides. In certain embodiments the method of the first aspect may involve determining the methylation level at least two and no more than four CpG dinucleotides. In certain embodiments the method of the first aspect may involve determining the methylation level at least two and no more than five CpG dinucleotides. In certain embodiments the method of the first aspect may involve determining the methylation level at least two and no more than six CpG dinucleotides. In certain embodiments the method of the first aspect may involve determining the methylation level at least two and no more than seven CpG dinucleotides. In certain embodiments the method of the first aspect may involve determining the methylation level at least two and no more than eight CpG dinucleotides. In certain embodiments the method of the first aspect may involve determining the methylation level at least two and no more than nine CpG dinucleotides. In certain embodiments the method of the first aspect may involve determining the methylation level at least two and no more than ten CpG dinucleotides.

Certain embodiments relate to a method of estimating the chronological age of human individuals, which comprises the method of the first aspect. For example, a method of estimating the chronological age of human individuals comprises 1) determining a methylation level of at least two CpG-dinucleotides of human skin cells; 2) determining the biological age of said skin cells by comparing said determined methylation level with empirically determined data representing a correlation between the methylation level of said CpG-nucleotides and the chronological age of at least one human individual; and 3) estimating the chronological age.

In certain embodiments, the method of the first aspect further comprises the step of estimating the chronological age of human individuals. The correlation between the biological age and chronological age is high using the method according to the invention, wherein the difference between the biological age and chronological age, the mean absolute error (MAE) determined by the method described herein, is no more than about 7 years, no more than about 6 years, no more than about 5.5 years, no more than about 5 years, no more than about 4.5 years, no more than about 4 years, no more than about 3.5 years, no more than about 3 years, no more than about 2 years, no more than about 1.5 years, no more than about 1 year, no more than about 0.5 years, no more than 0 years.

The difference in biological age and chronological age may be determined for a single individual. The deviation of a single data point from the best fit line superimposed onto the data points from all individuals can vary from about 0 years to about 15 years. For example, the deviation of a single data point from the best fit line would be about 0 years, about 1 year, about 2 years, about 3 years, about 4 years about 5 years, about 6 years, about 7 years, about 8 years, about 9 years, about 10 years, about 11 years, about 12 years, about 13 years, about 14 years, about 15 years. This is an indication of the health status of the human individual and the human skin.

In certain embodiments, the biological age of an unknown individual is determined as described above. From this biological age, the chronological age of the individual is estimated by applying the expected MAE for the CpG data points used.

In certain embodiments the correlation between the biological age and the chronological age is better than expected when using a method wherein the methylation levels for two CpG dinucleotides is determined. The correlation between the biological age and the chronological age may be further improved by additionally determining methylation levels in further CpG dinucleotides, such as, for example, in a total of three CpG dinucleotides, or in a total of four CpG dinucleotides, or in a total of five CpG dinucleotides, or in a total of six CpG dinucleotides, or in a total of seven CpG dinucleotides, or in a total of eight CpG dinucleotides, or in a total of nine CpG dinucleotides, or in a total of ten CpG dinucleotides. It is noted, however, that the methods using the methylation level of two CpG nucleotides lead to results which are broadly acceptable in practice, while the consideration of the methylation levels of additional CpG dinucleotides may lead to an improvement of the correlation, wherein this improvement, however, is reduced with every additional CpG dinucleotide considered.

In certain embodiments, the biological age and chronological age are used to calculate a value h, which is indicative of the health of a human individual, and the human skin. In certain embodiments, the value h is calculated according to formula (I)

h=biological age−chronological age (I)

Certain embodiments relate to a method of evaluating the health of an individual, which comprises the method of the first aspect and determining the value h. For example, a method of evaluating the health of an individual comprises 1) determining a methylation level of at least two CpG-dinucleotides of human skin cells; 2) determining the biological age of said skin cells by comparing said determined methylation level with empirically determined data representing a correlation between the methylation level of said CpG-nucleotides and the chronological age of at least one human individual; 3) estimating the chronological age; and 4) subtracting the biological age from the chronological age to determining the value h.

Without wishing to be bound by theory it is considered that if the value for h obtained from formula (I) is positive, the human individual is in poor health. In this case, a larger h number is indicative of the health of the human individual being worse than a smaller h number. If the value of h obtained from formula (I) is negative, it may be assumed that the individual is in good health.

If the value for h is positive, a reduction of this value may be desired, thereby improving the health of the individual. The h value may be reduced by a number of means, such as exercising, eating healthily, sleeping the right amount, drinking enough water and/or avoiding stress.

The h value may also be reduced by applying active agents onto the skin cells in the form of pharmaceutical and/or cosmetic agents. The active agent may contact the skin cells in vitro or in viva Using the in vitro method, the active agent is added to the culture medium of skin cells. Using the in vivo method, the skin cells of a human individual may be contacted with the active agent using topical, subcutaneous or intradermal administration.

An “active agent” as used herein is any agent that has a therapeutic effect and/or cosmetic effect on the individual. A therapeutic effect is the treatment and/or prevention of a disease. A cosmetic effect is the improvement of appearance, such as treating and/or preventing the signs of molecular aging. Signs of molecular aging are, for example, stem cell exhaustion, altered intercellular communication, genomic instability, telomere attrition, epigenetic alterations, loss of proteostasis, deregulated nutrient sensing, mitochondrial dysfunction, cellular senescence and changed proliferative capacity. In certain embodiments the active agent is a cosmetic agent. A cosmetic agent when applied to an individual may promote attractiveness, alter appearance, beautify and/or cleanse. The cosmetic agent may also prevent and/or treat the signs of phenotypical aging of human skin are for example wrinkle formation, pale complexion, reduced wound healing capacity, loss of elasticity and tightness.

In certain embodiments, the biological age is determined before and after contacting the skin cells with an active agent. For example, the biological age is determined, the skin cells are contacted with an active agent, followed by determining the biological age. The biological age before and after contacting the skin cells with an active agent are compared. A reduction in the biological age after treatment indicates a therapeutic effect and/or cosmetic effect of the active agent. The time between contacting the skin cells with an active agent and determining the biological age may be varied. The steps of contacting the skin cells with an active agent and determining the biological age may be repeated to provide information about the effect of the active agent on the skin cells over time. This method may have the advantage of being a fast and cost effective way to identify active cosmetic and/or pharmaceutical compounds and/or extracts.

In certain embodiments, the method according to the invention may have one or more of the following effects:
increased accuracy of estimation of chronological age from biological age;
increased reliability of correlation between chronological age and biological age;
reduced cost of determining the biological age;
reduced time required to determine the biological age;
efficient method to determine the effect of pharmaceutical or cosmetic agents on biological age.

It should be noted that the present invention may comprise any combination of the features and/or limitations referred to herein, except for combinations of such features which are mutually exclusive. The foregoing description is directed to particular embodiments of the present invention for the purpose of illustrating it. It will be apparent, however, to one skilled in the art, that many modifications and variations to the embodiments described herein are possible. All such modifications and variations are intended to be within the scope of the present invention, as defined in the appended claims.

For the avoidance of doubt, the present application is directed to subject-matter described in the following numbered paragraphs.
1. A method for determining the biological age of human skin comprising:
   a) providing human skin cells;
   b) determining a methylation level of at least two CpG-dinucleotides of a specific region of at least one chromosome of said skin cells; and
   c) determining the biological age of said skin cells by comparing said determined methylation level with empirically determined data representing a correlation between the methylation level of said CpG-nucleotide and the chronological age of at least one human individual.
2. The method of paragraph 1, wherein the skin cells are harvested from a human individual.
3. The method of paragraph 1, wherein the skin cells are cultured in vitro.
4. The method according to any preceding numbered paragraph, wherein the methylation of two CpG-dinucleotides of a specific region of one or two chromosomes of said human skin cells is determined.
5. The method according to any one of the preceding paragraphs, wherein each specific region of the chromosome comprises at least one nucleotide sequence selected from SEQ ID NO: 1 to SEQ ID NO: 205.
6. The method according to any preceding paragraph, wherein the one or two of the nucleotide sequences are selected from SEQ ID NO: 51 and SEQ ID NO: 110.
7. The method according to any preceding paragraph, wherein at least one of the nucleotide sequences are selected from SEQ ID NO: 91, SEQ ID NO: 179, SEQ ID NO: 202, SEQ ID NO: 203; SEQ ID NO: 204; and SEQ ID NO: 205.
8. The method of any one of the preceding paragraphs, wherein said data comprises at least one linear regression equation, preferably one equation which comprises at least two linear regressions.
9. The method according to any preceding paragraph, further comprising the step of estimating the chronological age of the human individual.
10. The method of paragraph 9, wherein the chronological age is estimated to be within 5.25 years of the biological age.
11. The method according to any preceding paragraph, further comprising the step of calculating a value h indicative of the health of a human individual, where h is calculated based on the biological age and chronological age.
12. The method according to paragraph 11, wherein h is calculated based on the difference between biological age and chronological age.
13. The method according to any one of the proceeding claims further comprising the step of contacting the skin cells with an active agent.
14. The method according to paragraph 13, wherein the biological age is determined before and after contacting the skin cells with an active agent.
15. A method of testing an active agent comprising the method of any one of paragraphs 1 to 10, further comprising the steps of:
   d) contacting the skin cells of step a) with an active agent;
   e) determining the biological age of the skin cells of step d) according to the method of any one of paragraphs 1 to 10; and
   f) comparing the biological age determined in steps a) to c) with the biological age determined in step e).
16. The method of paragraph 15, wherein step d) is carried out either in vivo or in vitro.
17. The method of paragraph 15 or 16, wherein the active agent is a cosmetic agent and/or a therapeutic agent.
18. A method of any one of paragraphs 15 to 17 to identify an active agent that prevents and/or treats the signs of phenotypical aging of human skin.
19. Use of a nucleic acid molecule for determining the biological age of human skin according to the method of any one of paragraphs 1 to 18, wherein said nucleic acid molecule comprises at least one nucleotide sequence selected from the group consisting of:
   d) a nucleotide sequence comprising at least one sequence selected from the group consisting of SEQ ID NO: 1 to SEQ ID NO: 205;
   e) a nucleotide sequence which differs from the nucleotide sequence of a) by replacement of at most 10% of the nucleotides, preferably at most 5 of the nucleotides, but for said CpG-dinucleotide;
   f) a nucleotide sequence which corresponds to the complementary strand of the nucleotide sequence a) or b).
20. The use of paragraph 19, wherein two nucleotide sequences are selected.
21. Computer-readable medium having stored computer-executable instructions for causing a computer to perform a method for determining the biological age of human skin comprising:
   d) inputting at least two values and no more than 10 of a determined methylation level of at least two and no more than 10 CpG-dinucleotides of a specific region of at least one chromosome of said skin cells;
   e) comparing said value of the determined methylation level with stored data representing a correlation between the methylation level of said CpG-dinucleotide and the chronological age of at least one human individual; and
   f) displaying the biological age.

22. Computer-readable medium according to paragraph 21, wherein said stored data comprise at least one linear regression equation, preferable one equation which comprises at least two linear regressions.
23. A kit for determining the biological age of human skin according to the method of any one of the paragraphs 1 to 10, comprising at least one oligonucleotide primer for amplifying and/or sequencing at least two CpG-nucleotides of at least one nucleotide sequence selected from the group consisting of SEQ ID NO: 1 to SEQ ID NO: 205.

### EXAMPLES

### Skin Samples and Patient Group

Data of 108 epidermal samples (Bormann, Aging Cell 2016) from DNA-methylation arrays (Illumina 450k BeadChip) was collected. To obtain the age-associated CpG sites, the correlation coefficient (r) between the chronological age of the samples and corresponding beta-values for each CpG site were calculated. After pre-filtering, 225 age-associated CpG sites (r>0.8 or r<-0.8) were selected. A multivariable linear model was trained using the complete set of 225 CpG sites.

In order to find suitable CpG combinations, the complete sample set was divided into training set and test set. Single CpGs or CpG combinations of at least two CpGs were chosen due to their prediction accuracy.

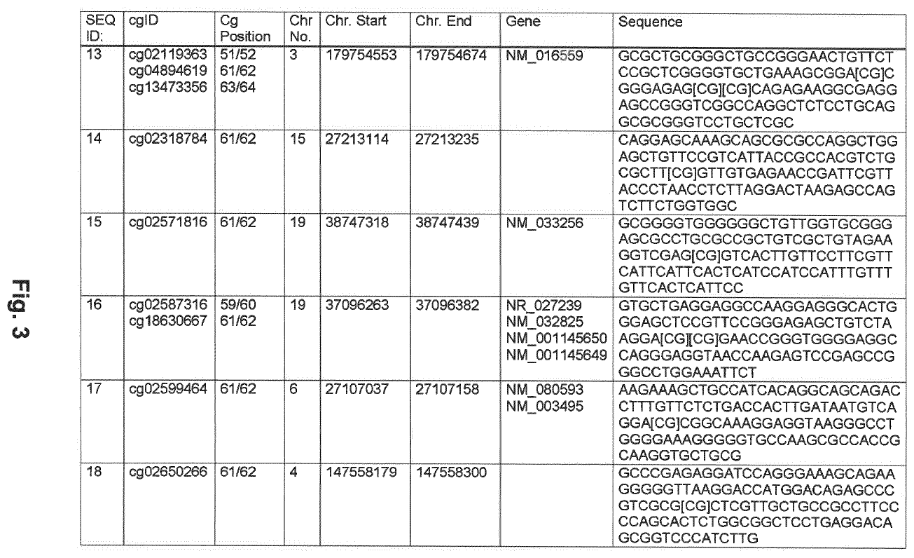

A correlation of the age dependent change in methylation is shown in FIG. 2 for two CpGs markers: cg06335867 and cg13848598.

Skin samples were collected from healthy volunteers aged between 23 and 54 years using suction blistering. For whole epidermis samples, the epidermal parts of the skin were separated from the dermal parts by heat split. To isolate keratinocytes, epidermis was separated from dermis by dispase II (Roche 04942078001) digestion, and keratinocyte were isolated after trypsin (Life Technologies #25200056) treatment.

### DNA Isolation and Bisulfite Conversion

The DNA was isolated using the QIAmp DNA Investigator Kit (Qiagen #56504). The bisulfite conversion of 500 ng of DNA was carried out using in vitro cultivated human keratinocytes and ex vivo human epidermis samples with an EpiTect Bisulfite Kit (Qiagen #59104). The DNA was converted by following manufacturer's protocol, but wherein an incubation time of 4h 55 min was used instead of 2h 55 min.

### Amplicon PCR and Sequencing

To analyse the CpGs, amplicons were generated using PCR. The PCR was carried out using the bisulfite converted DNA and the PyroMark PCR Kit (Qiagen #978703). The PCR composition is shown in Table 1. The PCR was performed with an annealing temperature of 55° C.

TABLE 1
PCR composition

| Component¹ | Amount |
|---|---|
| Bisulfite converted DNA | 50 ng in 2 μl of water |
| Mastermix (2x) | 12.5 μl |
| Coral loading dye | 2.5 μl |
| MgCl₂ (25 mM) | 1 μl |
| Forward primer (10 μM) | 0.5 μl |
| Reverse primer (10 μM) | 0.5 μl |
| water | 6 μl |

¹MgCl₂, forward primer and reverse primer were used as aqueous solutions.

The PCR fragments were unravelled in a 1% agrosegel in TAE buffer solution and cut out of the gel. The amplicons were purified using a Qiaquick gel extraction kit (Qiagen #28706).

The DNA sequencing was carried out using a 454-sequencing approach (Roche) according to the manufacturer's protocol.

The extent of methylation was determined from the proportion of methylated and unmethylated Reads.

### Calculating the Biological Age

To calculate the biological age, the proportion of methylation at a CpG-Locus was determined, to provide the beta-value. The average beta-value (β) was calculated for each CpG locus and each sample. It was then converted to the m-value (m) using formula (1):

m=log 2(β/1−β) (1)

The m-value may then be used in, for example, in one of the stated equations (2) to (4) for cg06335867 and cg13848598:

Biological age=70.002+8.992*m_{cg06335867}+12.998*m_{cg13848598} (2)

Biological age=68.785+7.346*m_{cg06335867}+15.865*m_{cg13848598} (³)

Biological age=60.372+6.923*m_{cg06335867}+12.904*m_{cg13848598} (4)

Other examples of equations that could be used for other CpG-dinucleotides include following:

One of the stated equations (5) to (7) for cg13848598 and cg25015733

Biological age=49.9135+32.3373*m_{cg13848598}−4.3823*m_{cg25015733} (⁵)

Biological age=40.0731+11.7548*m_{cg13848598}−3.7269*m_{cg25015733} (6)

Biological age=66.5409+6.0015*m_{cg13848598}+0.5269*m_{cg25015733} (⁷)

One of the stated equations (8) to (10) for cg06335867 and cg12494373

Biological age=77.9930+13.8956*m_{cg06335867}−11.1632*m_{cg12494373} (⁸)

Biological age=45.505+4.103*m_{cg06335867}−3.89*m_{cg12494373} (⁹)

Biological age=73.506+5.203*m_{cg06335867}−3.63*m_{cg12494373} (10)

One of the stated equations (11) to (13) for cg23368787 and cg03271893

Biological age=49.0849+23.5911*m_{cg23368787}−1.7116*m_{cg03271893} (11)

Biological age=34.928+8.129*m_{cg23368787}−5.868*m_{cg03271893} (12)

Biological age=64.454+8.149*m_{cg23368787}−3.872*m_{cg03271893} (13)

One of the stated equations (14) to (16) for cg11084334 and cg24217948

Biological age=69.9697+24.0016*m_{cg11084334}−11.8749*m_{cg24217948} (14)

Biological age=54.792+11.685*m_{cg11084334}−6.465*m_{cg 24217948} (15)

Biological age=50.985+3.226*m_{cg11084334}+6.147*m_{cg24217948} (16)

### Example 1

Skin samples were collected from 6 individuals an analysed according to the methods provided above. The CpG-nucleotides analysed were cg06335867 and cg13848598. The following calculation of biological age were carried out using equation (2) and compared to the chronological age.

### Subiect 1

Chronological age: 30 years old
Marker cg06335867:

Measured β−value (cg06335867): 0.1702

Calculated m−value (cg06335867)=log 2 (0.1702/1−0.1702)=−2.2855326
Marker cg13848598:

Measured β−value (cg13848598): 0.25

Calculated m−value (cg13848598)=log 2 (0.25/1−0.25)=−1.5849625
Biological age:

70.002+8.992×(−2.2855326)+12.998×(−1.5849625)=28.85 years old

Difference between chronological and biological age: 1.15 years.

### Subject 2

Chronological age: 35 years old
Marker cg06335867:

Measured β−value (cg06335867): 0.2286

Calculated m−value (cg06335867)=log 2 (0.2286/1−0.2286)=−1,7546537
Marker cg13848598:

Measured β−value (cg13848598): 0.3775

Calculated m−value (cg13848598)=log 2 (0.3775/1−0.3775)=−0.7215972
Biological age:

70.002+8.992×(−1.7546537)+12.998×(−0.7215972)=44.84 years old

Difference between chronological and biological age: 9.84 years.

### Subject 3

Chronological age: 57 years old
Marker cg06335867:

Measured β−value (cg06335867): 0.1681

Calculated m−value (cg06335867)=log 2 (0,1681/1−0.1681)=−2.3070904
Marker cg13848598:

Measured β−value (cg13848598): 0.5514

Calculated m−value (cg13848598)=log 2 (0.5514/1−0.5514)=0.2976696
Biological age:

70.002+8.992×(−2.3070904)+12.998×(0.2976696)=53.13 years hold

Difference between chronological and biological age: 3.87 years.
Subject 4
Chronological age: 72 years old
Marker cg06335867:

Measured β−value (cg06335867): 0.3262

Calculated m−value (cg06335867)=log 2 (0.3262/1−0.3262)=−1.0465636
Marker cg13848598:

Measured β−value (cg13848598): 0.6071

Calculated m−value (cg13848598)=log 2 (0.6071/1−0.6071)=0.62777201
Biological age:

70.002+8.992×(−1.0465636)+12.998×(0.62777201)=68.75 years hold

Difference between chronological and biological age: 3.25 years.

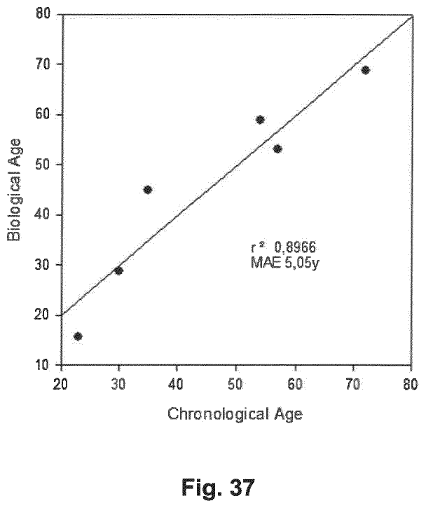

The calculated biological age was plotted against the chronological age of each one of six individuals, as seen in FIG. 37. The mean absolute error across these data points was 5.05 years, which demonstrates a high degree of correlation between the calculated biological age according to the invention and chronological age. Furthermore, a good correlation was seen at all ages. Since the method predicts biological age with high accuracy over all samples, deviating biological age is indicative of the health status of a single individual.

1.-21. (canceled)

22. A method for determining the biological age of human skin, wherein the method comprises:
(a) determining a methylation level of at least two CpG-dinucleotides of human skin cells; and
(b) determining the biological age of the skin cells by comparing the determined methylation level with empirically determined data representing a correlation between the methylation level of the CpG-nucleotides and a chronological age of at least one human individual.

23. The method of claim 22, wherein each CpG-dinucleotide whose methylation level is determined is a part of a nucleotide sequence selected from SEQ ID NO: 1 to SEQ ID NO: 205.

24. The method of claim 22, wherein the methylation levels of two CpG-dinucleotides of human skin cells are determined.

25. The method of claim 22, wherein at least one of the at least two CpG-dinucleotides whose methylation level is determined is a part of a nucleotide sequence selected from SEQ ID NO: 51, SEQ ID NO: 91, SEQ ID NO: 110, SEQ ID NO: 179, SEQ ID NO: 202, SEQ ID NO: 203, SEQ ID NO: 204, and SEQ ID NO: 205.

26. The method of claim 22, wherein each CpG-dinucleotide whose methylation level is determined is a part of a nucleotide sequence selected from SEQ ID NO: 51, SEQ ID NO: 91, SEQ ID NO: 110, SEQ ID NO: 179, SEQ ID NO: 202, SEQ ID NO: 203, SEQ ID NO: 204, and SEQ ID NO: 205.

27. The method of claim 22, wherein said empirically determined data comprises at least one linear regression equation.

28. The method of claim 27, wherein said empirically determined data comprises one equation which comprises at least two linear regressions.

29. The method of claim 22, wherein the method further comprises estimating the chronological age of the human individual.

30. The method of claim 29, wherein the chronological age is estimated to be within 5.25 years of the biological age.

31. The method of claim 22, wherein the method further comprises calculating a value h which indicative of a health of a human individual, h being calculated based on the biological age and a chronological age.

32. The method of claim 31, wherein h is calculated based on a difference between biological age and chronological age.

33. The method of claim 22, wherein the method further comprises contacting the skin cells with an active agent.

34. The method of claim 33, wherein the biological age is determined before and after contacting the skin cells with the active agent.

35. A method of testing an active agent, wherein the method comprises
(a) determining a methylation level of at least two CpG-dinucleotides of human skin cells;
(b) determining a biological age of the skin cells by comparing the determined methylation level with empirically determined data representing a correlation between the methylation level of the CpG-nucleotides and a chronological age of at least one human individual
(c) contacting the skin cells with the active agent;
(d) determining the biological age of the skin cells obtained in (c) according to the (b); and
(e) comparing the biological age determined according to (a) and (b) with the biological age determined in (d).

36. The method of claim 35, wherein the active agent is a cosmetic agent and/or a therapeutic agent.

37. The method of claim 35, wherein the method is used to identify an active agent that prevents and/or treats signs of phenotypical aging of human skin.

38. The method of claim 22, wherein the method comprises employing a nucleic acid molecule that comprises at least one nucleotide sequence selected from
(i) a nucleotide sequence comprising at least one sequence selected SEQ ID NO: 1 to SEQ ID NO: 205;
(ii) a nucleotide sequence which differs from the nucleotide sequence of (i) by replacement of at most 10% of nucleotides, except for said CpG-dinucleotide;
(iii) a nucleotide sequence which corresponds to a complementary strand of the nucleotide sequences (i) and/or (ii).

39. A computer-readable medium, wherein the medium comprises stored therein computer-executable instructions for causing a computer to perform a method for determining a biological age of human skin, which method comprises:
a) inputting at least two values of a determined methylation level of at least two CpG-dinucleotides of human skin cells;
b) comparing the value of the determined methylation level with stored data representing a correlation between the determined methylation level of said CpG-dinucleotide and the chronological age of at least one human individual; and
c) displaying the biological age.

40. The computer-readable medium of claim 39, wherein the stored data comprise at least one linear regression equation.

41. A kit for carrying out the method of claim 22, wherein the kit comprises at least one oligonucleotide primer for amplifying and/or sequencing at least two CpG-nucleotides of at least one nucleotide sequence selected from SEQ ID NO: 1 to SEQ ID NO: 205.
